(19) United States
(12) Patent Application Publication
(10) Pub. No.: US 2008/0051597 A1
(43) Pub. Date: Feb. 28, 2008

(54) Process for Producing 2-Methyladamantan-2-Yl (Meth)Acrylate and 2-Methyladamantan-2-Yl (Meth)Acrylate

(75) Inventors: Hidekichi Hashimoto, Fukuoka (JP); Kaoru Hamashima, Fukuoka (JP); Shigenori Kawamura, Fukuoka (JP); Toshiyuki Suzuki, Fukuoka (JP); Yuji Ohgomori, Kanagawa (JP)

Correspondence Address: OBLON, SPIVAK, MCCLELLAND MAIER & NEUSTADT, P.C., 1940 DUKE STREET, ALEXANDRIA, VA, 22314, US

(73) Assignee: Mitsubishi Chemical Corporation, Minato-ku (JP)

(21) Appl. No.: 11/573,843

(22) PCT Filed: Aug. 19, 2004
(86) PCT No.: PCT/JP04/12227

§ 371 Date: Nov. 9, 2007

(51) Int. Cl. C07C 69/74

(52) U.S. Cl. 560/116

(57) ABSTRACT

An object of the present invention is to provide a process for purifying 2-methyladamantan-2-yl(meth)acrylate with high separation efficiency and an industrially advantageous preparation process of 2-methyladamantan-2-yl(meth)acrylate. The present invention pertains to a preparation process of 2-methyladamantan-2-yl(meth)acrylate which comprises reacting 2-methyleneadamantane and (meth)acrylic acid by an acid catalyst to obtain a reaction mixture, and then separating 2-methyleneadamantane from the reaction mixture to obtain 2-methyladamantan-2-yl(meth)acrylate, wherein the reaction mixture is distilled in the presence of an organic compound having a boiling point, under atmospheric pressure, of 150° C. or greater but not greater than 260° C.

### TECHNICAL FIELD

The present invention relates to a preparation process of 2-methyladamantan-2-yl(meth)acrylate.

More specifically, the present invention relates to a process for obtaining highly-pure 2-methyladamantan-2-yl(meth)acrylate by reacting (meth)acrylic acid with 2-methyleneadamantane by using an acid catalyst and then purifying the resulting reaction mixture. The 2-methyladamantan-2-yl(meth)acrylate available by the present invention is useful as fine chemicals such as pharmaceuticals, agrichemicals and Information and electronic raw materials.

### BACKGROUND ART

There are some reports on the preparation processes of 2-methyladamantan-2-yl(meth)acrylate. A preparation process of 2-methyladamantan-2-yl(meth)acrylate which comprises reacting (meth)acrylic acid with 2-methyleneadamantane in the presence of an acid catalyst, and separating 2-methyleneadamantane from the resulting mixture is reported (refer to Patent Document 1). This process uses neither acid halide nor organic amine, so that it has the advantage of reducing an incorporation amount of a halogen or organic amine mixed into 2-methyladamantan-2-yl(meth)acrylate.

Also, a process of causing a carboxylic acid to act on 2-methyladamantanol is disclosed as a preparation process of 2-methyleneadamantane which is a raw material (refer to Patent Document 2). When 2-methyleneadamantane is reacted with (meth)acrylic acid in the presence of an acid catalyst, 2-methyleneadamantane remains after the reaction because of limitations in equilibrium, thus is necessary to be separated and removed. As a process for separating 2-methyleneadamantane, a process of distilling it in the presence of water is disclosed (refer to Patent Document 3).

As another reaction route, a process of reacting (meth)acrylic anhydride or (meth)acrylic halide with 2-methyladamantanol, thereby obtaining 2-methyladamantan-2-yl(meth)acrylate is known. A mixture obtained by such a reaction contains impurities such as adamantane, 2-methyleneadamantane, 2-adamantanone and 2-methyl-2-adamantanol. A process of removing these sublimable impurities by distilling the reaction mixture in the presence of a compound having a lower boiling point than the target compound, such as cyclic amide or cyclic urea is disclosed (refer to Patent Document 4).
[Patent Document 1] JP-A-2002-284739
[Patent Document 2] JP-A-2002-47216
[Patent Document 3] JP-A-2002-97171
[Patent Document 4] JP-A-2001-97893

### DISCLOSURE OF THE INVENTION

Known processes however pose some problems when they are carried out industrially. They do not include any process capable of efficiently removing 2-methyleneadamantane from a mixture which has been obtained by reacting (meth)acrylic acid with 2-methyleneadamantane in the presence of an acid catalyst. For example, there is room for improvement, from the viewpoint of separation efficiency, in the process as disclosed in Patent Document 3, because when distillation in the presence of water is performed, a difference in the boiling point from 2-methyleneadamantane which is to be separated is large. In the process as described in Patent Document 4, the mixture to be separated has a quite different composition presumably because of a difference in the reaction route. Use of a cyclic amide or cyclic urea is considered to be preferred in order to prevent adhesion of a sublimable compound such as 2-adamantanone or 2-methyl-2-adamantanol, which is, however, not optimum condition for the separation of 2-methyleneadamantane. In addition, use of a compound having a lower boiling point than that of the target organic compound as a distillation aid is disclosed in this Patent Documents but so many solvents fall into this category and this makes it difficult to select a distillation aid capable of effectively removing 2-methyleneadamantane from a mixture of 2-methyleneadamantane and 2-methyladamantan-2-yl(meth)acrylate.

With the forgoing problems in view, the present inventors have carried out an extensive investigation on a process of separating and removing 2-methyleneadamantane from a mixture obtained by reacting (meth)acrylic acid with 2-methyleneadamantane by an acid catalyst and, completed the present invention.

In a first aspect of the present invention, there is thus provided a process for preparing 2-methyladamantan-2-yl(meth)acrylate, which comprises: reacting 2-methyleneadamantane and (meth)acrylic acid by an acid catalyst to obtain a reaction mixture; and separating 2-methyleneadamantane from the reaction mixture to obtain 2-methyladamantan-2-yl(meth)acrylate, wherein the reaction mixture is distilled in the presence of an organic compound having a boiling point, under atmospheric pressure, of 150° C. or greater but not greater than 260° C. and immiscible with water.

In a second aspect of the present invention, there is also provided a process for preparing 2-methyladamantan-2-yl(meth)acrylate, which comprises the following steps:

(a) a step of reacting 2-methyleneadamantane with (meth)acrylic acid by an acid catalyst to obtain a mixture containing 2-methyleneadamantane and 2-methyladamantan-2-yl(meth)acrylate;

(b) a step of distilling the reaction mixture in the presence of an organic compound having a boiling point, under atmospheric pressure, of 150° C. or greater but not greater than 260° C. and immiscible with water, to distill off a fraction comprising 2-methyleneadamantane as a main component from the top of a distillation column and to obtain a fraction comprising 2-methyladamantan-2-yl(meth)acrylate as a main component from the bottom of the distillation column; and

(c) re-distilling the fraction obtained from the bottom of the distillation column and comprising 2-methyladamantan-2-yl(meth)acrylate as a main component to remove the organic compound having a boiling point of 150° C. or greater but not greater than 260° C., 2-methyleneadamantane and a portion of 2-methyladamantan-2-yl(meth)acrylate from the top of the distillation column to reduce the amount of 2-methyleneadamantane present in 2-methyladamantan-2-yl(meth)acrylate to 1 wt. % or less.

In a third aspect of the present invention, there is also provided a process for preparing 2-methyladamantan-2-yl(meth)acrylate, which comprises: reacting 2-methyleneadamantane and (meth)acrylic acid in the presence of an organic compound having a boiling point, under atmospheric pressure, of 150° C. or greater but not greater than 260° C. and immiscible with water by an acid catalyst to obtain a reaction mixture; and separating 2-methyladamantane from the reaction mixture by distillation to obtain 2-methyladamantan-2-yl(meth)acrylate.

In a fourth aspect of the present invention, there is also provided a process for preparing 2-methyladamantan-2-yl(meth)acrylate, which comprises the following steps:

(a) a step of reacting 2-methyleneadamantane with (meth)acrylic acid by an acid catalyst to obtain a mixture containing 2-methyleneadamantane and 2-methyladamantan-2-yl(meth)acrylate;

(b) a step of distilling the reaction mixture in the presence of an organic compound having a boiling point, under atmospheric pressure, of 150° C. or greater but not greater than 260° C. and immiscible with water, to distill off a fraction comprising 2-methyleneadamantane as a main component from the top of a distillation column and to obtain a fraction comprising 2-methyladamantan-2-yl(meth)acrylate as a main component from the bottom of the distillation column; and

(c) a step of recycling, to the step (a), at least a portion of the fraction containing 2-methyleneadamantane and the organic compound having a boiling point, under atmospheric pressure, of 150° C. or greater but not greater than 260° C. and immiscible with water.

In a fifth aspect of the present invention, there is also provided 2-methyladamantan-2-yl(meth)acrylate comprising 2-methyleneadamantane in an amount of 1 wt. % or less.

The present invention makes it possible to obtain, from a mixture obtained by reacting 2-methyleneadamantane and (meth)acrylic acid in the presence of an acid catalyst, the target 2-methyladamantan-2-yl(meth)acrylate with high separation efficiency.

### BEST MODE FOR CARRYING OUT THE INVENTION

The present invention will hereinafter be described specifically.

<Preparation of 2-methyleneadamantane>

A known process can be employed for the preparation of 2-methyleneadamantane to be used in the present invention, but one preferable process to obtain it is dehydration reaction of 2-methyladamantan-2-ol. This dehydration reaction is carried out preferably in the presence of a carboxylic acid.

No particular limitation is imposed on the carboxylic acid to be used in this reaction. Specific examples include saturated or unsaturated aliphatic carboxylic acids such as acetic acid, propionic acid, butyric acid, valeric acid, acrylic acid and methacrylic acid; and saturated or unsaturated aromatic carboxylic acids such as benzoic acid, phenylacetic acid, naphthalenecarboxylic acid and cinnamic acid. Any of them is usable. Dibasic acids such as adipic acid and benzenedicarboxylic acid are also usable. Of these, acrylic acid, methacrylic acid, acetic acid and the like are preferred. Although the amount of the carboxylic acid varies, depending on the kind of it, it is usually 0.1 molar equivalent or greater but not greater than 5 times molar equivalent relative to a raw material 2-alkyladamantan-2-ol. The dehydration reaction is preferably carried out in the presence of an acid catalyst such as mineral acids and organic sulfonic acids.

A solvent stable in the dehydration reaction system is also usable. Since this dehydration reaction is an equilibrium reaction, it is advantageous to use a solvent azeotropic with water to be produced with the progress of the reaction and remove the thus-produced water out of the reaction system by azeotropy with the solvent. Specific examples of such a solvent include aliphatic hydrocarbons such as hexane and heptane, aromatic hydrocarbons such as benzene, toluene and xylene, and halogenated hydrocarbons such as chloroform. The azeotropic removal of the resulting water out of the system may be effected by using a water droplet separator such as Dean Stark trap and returning the solvent which has remained after the removal of water to the reaction system. Although the amount of the solvent cannot be determined in a wholesale manner and it may be added as desired, the lower limit is usually 0.01 time, preferably 0.1 time and the upper limit is usually 1000 times, preferably 500 times, each in terms of the weight of the raw material 2-alkyladamantan-2-ol.

The dehydration reaction is preferably performed at a predetermined temperature for a predetermined time, for example, by charging, in a reactor, a 2-alkyladamantan-2-ol as a raw material and a carboxylic acid as a catalyst, and, if necessary, an acid catalyst and a solvent preferably under stirring and preferably while distilling off water produced by the reaction out of the system.

The 2-methyladamantan-2-ol to be subjected to the dehydration reaction can be easily prepared, for example, by the reaction between 2-adamantanone and a Grignard reagent such as methyl magnesium chloride or methyl magnesium bromide.

<Reaction Between 2-methyleneadamantane and (meth)acrylic acid>

A known process can be employed for the reaction of (meth)acrylic acid with 2-methyleneadamantane by using an acid catalyst. It is, for example, carried out by charging a reactor with 2-methyleneadamantane and (meth)acrylic acid as raw materials and an acid catalyst, and, if necessary, a solvent and reacting them at a predetermined temperature for a predetermined time preferably while stirring. In the process of the present invention, the reaction may be effected either in a solventless manner or in a solvent which is stable in the reaction system. When a Bronsted acid is used as the catalyst, aprotic solvents are usable. Examples thereof include aliphatic saturated hydrocarbons such as hexane, heptane, octane and decane; aromatic hydrocarbons such as benzene, toluene and xylene, ethers such as diethyl ether, dipropyl ether, dibutyl ether, tetrahydrofuran and dioxane; lactones such as γ-butyrolactone and δ-valerolactone; and halogenated hydrocarbons such as dichloromethane, dichloroethane, chloroform and chlorobenzene. As a protic solvent, (meth)acrylic acid serving also as a reaction substrate is usable. It is also possible to use, as a portion or whole portion of the reaction solvent, the below-described organic compound which is caused to exist at the time of distillation, has a boiling point, under atmospheric pressure, of 150° C. or greater but not greater than 260° C., and is immiscible with water.

Solvents suited for use when a Lewis acid is used as the catalyst are similar to those employed when the above-described Bronsted acid is used as the catalyst except for the omission of solvents such as ethers and lactones showing strong coordination to the catalyst. Although the amount of the solvent cannot be determined in a wholesale manner and it may be added as needed, the lower limit is usually 0.01 time, preferably 0.1 time and the upper limit is usually 1000 times, preferably 500 times, each in terms of the weight of the raw material 2-methyleneadamantane.

The addition reaction of the present invention is an exothermic reaction and as is apparent from the equilibrium constant, reaction at a lower temperature is advantageous for improvement of the yield. In fact, when a Lewis acid is used as the catalyst, the target reaction proceeds very smoothly even at a reaction temperature lower than the room temperature, resulting in the preparation of a tertiary alkyl ester of (meth)acrylic acid in a short time and in a high yield.

The lower limit of the reaction temperature is usually −80° C., preferably −50° C., while the upper limit of the reaction temperature is usually 200° C., preferably 100° C.

The optimum reaction time varies, depending on the reaction temperature, but the lower limit is usually 0.01 hour, preferably 0.1 hour, while the upper limit is usually 50 hours, preferably 20 hours.

If necessary, another additive, for example, an additive for suppressing the polymerization of (meth)acrylic acid may be caused to exist in the reaction system. In this reaction, for example, a 2-alkyladamantan-2-yl(meth)acrylate can be prepared by carrying out dehydration reaction of a 2-alkyladamantan-2-ol by using a Bronsted acid catalyst to obtain the corresponding 2-alkylideneadamantane, adding a predetermined amount of (meth)acrylic acid to the reaction mixture and then allowing the reaction mixture to stand at a low temperature, for example, room temperature. Alternatively, a 2-alkyladamantan-2-yl(meth)acrylate can be prepared at a higher efficiency by adding predetermined amounts of (meth)acrylic acid and a Lewis acid such as boron trifluoride to the above-described reaction mixture containing the 2-alkylidenadamantane and then allowing it to stand or stirring it. The present reaction can be carried out under batch operation, but it may also be carried out under continuous operation if a proper reactor is used.

<Distillation of a Mixture of 2-methyleneadamantane and 2-methyladamantan-2-yl(meth)acrylate>

The present invention is characterized in that the thus-obtained mixture mainly containing 2-methyleneadamantane and 2-methyladamantan-2-yl(meth)acrylate is distilled in the presence of an organic compound having a specific boiling point under atmospheric pressure and immiscible with water to efficiently separate 2-methyleneadamantane from the mixture. The term “immiscible with water” as used herein means that a separation into two layers is not occurred when the organic compound is mixed with water at a mixing ratio of 1:1.

The lower limit of boiling point under atmospheric pressure of the organic compound used in the above reaction has a boiling point, is 150° C. or greater, preferably 170° C. or greater, but the upper limit is not greater than 260° C., preferably not greater than 240° C., and a coagulation point thereof is 10° C. or less, preferably 0° C. or less, more preferably −10° C. or less, especially preferably −20° C. or less. The lower limit of the coagulation point is not particularly limited.

The organic compound having a boiling point exceeding the above-described range is not preferred because such an organic compound disturbs separation from 2-methyladamantyl methacrylate, while that having a boiling point less than the above-described range leads to an inefficiency because the amount thereof necessary for the removal of methyleneadamantane increases inevitably. The melting point of the organic compound must be such that the compound is in the form of a solution at the operation temperature of a condenser during distillation.

The solubility, at 0° C., of 2-methyleneadamantane in 100 g of the organic compound is 5 g or greater, preferably 20 g or greater, more preferably 50 g or greater, most preferably 70 g or greater. The organic compound permitting high solubility of 2-methyleneadamantane therein is preferred because even a small amount thereof is effective.

Moreover, use of a hydrocarbon compound as the organic compound is preferred because it has a smaller reactivity with the target compound, has higher stability during the distillation, and has less influence on the product compound even if a small amount of it remains therein.

Examples of the compound having such properties include aliphatic hydrocarbons such as decane, undecane, tridecane, tetradecane and dodecane and aromatic hydrocarbons such as ethyl toluene, butyl benzene and 5-t-butyl-m-xylene. Use of hydrocarbons having such properties enables efficient removal of 2-methyladamantane. Of these, aliphatic hydrocarbons are preferred, with aliphatic saturated hydrocarbons being more preferred.

The lower limit of the used amount organic compound is usually 0.5 time the weight or greater, preferably 2 times the weight or greater, and the upper limit is not greater than 20 times the weight, preferably not greater than 10 times the weight, each in terms of the weight of 2-methyleneadamantane. The entire amount of the organic compound may be added in advance to the reaction mixture. Alternatively, it is possible to add a portion of the organic compound in advance and then, add a remaining portion sequentially during the reaction. When the organic compound is used as a whole portion or a portion of the reaction solvent as described above, addition of it at the time of distillation is not required.

2-Methyladamantan-2-yl(meth)acrylate is a compound having a polymerizable functional group and is thermally instable. Distillation operation is performed usually at a low temperature under reduced pressure. The lower the pressure, the better. Distillation is performed usually at 20 mmHg or less, preferably 10 mmHg or less, still more preferably 5 mmHg or less. Distillation temperature varies, depending on the vacuum conditions, but is usually 170° C. or less, preferably 150° C. or less, especially preferably 130° C. or less. By this distillation operation, 2-methyleneadamantane and the organic compound which is caused to exist therewith and has a boiling point of 150° C. or greater but not greater than 260° C. can be distilled off together. The distillation operation of the present invention may be either batch distillation or continuous distillation. Since the target compound of the present invention is a thermally instable compound, continuous distillation is preferred in view of a short retention time at a heating zone and productivity. Examples of the continuous distillation include flash distillation and molecular distillation. As an industrial apparatus, a thin film distillation apparatus ordinarily usable for distillation of thermally instable compounds can be employed.

As one mode for carrying out the present invention, a separation and purification process by performing two-stage continuous distillation, which will be described below, can be exemplified.

After addition of the organic compound having a boiling point of 150° C. or greater but not greater than 260° C. and immiscible with water to the reaction mixture, a fraction comprising a mixture of 2-methyleneadamantane and the organic compound as a main component is removed by distillation from the top of the distillation column. By this operations 2-methyleneadamantane and the organic compound have been substantially removed from the solution obtained from the bottom of the distillation column.

Second distillation operation of the mixture comprising 2-methyladamantan-2-yl(meth)acrylate as a main component thus obtained is performed in order to further reduce the concentration of 2-methyleneadamantane and the organic compound. In this operation, the organic compound, 2-methyleneadamantane and a portion of 2-methyladamantan-2-yl(meth)acrylate are distilled off from the top of the distillation column so as to reduce the amount of 2-methyleneadamantane in the 2-methyladamantan-2-yl (meth)acrylate available from the bottom of the distillation column to 1 wt. % or less. The distillate obtained from the top of the distillation column in the second stage contains 2-methyladamantan-2-yl(meth)acrylate so that the distillate is recycled to the first-stage operation. The yield can be improved by repeating this operation.

In such a manner, 2-methyladamantan-2-yl(meth)acrylate from which 2-methyleneadamantane has been removed can be purified further by the distillation operation as needed.

<Recycling of 2-methyleneadamantane and Organic Compound to Reaction Step>

The fraction available from the top of the distillation column by the first-stage distillation operation contains large amounts of 2-methyleneadamantane and the organic compound having a boiling point of 150° C. or greater but not greater than 260° C. and immiscible with water. By the recycling use of such a fraction for esterification reaction, a portion of 2-methyleneadamantane can be converted into 2-methyladamantan-2-yl(meth)acrylate. Although this addition reaction is subject to equilibrium constraints, the recycling operation makes it possible to improve the consistent yield, and has an industrial advantage.

<2-methyladamantan-2-yl(meth)acrylate>

The amount of 2-methyleneadamantane contained in the 2-methyladamantan-2-yl(meth)acrylate of the present invention available by the above-described process is 1 wt. % or less. This amount can be measured by a known gas chromatography analysis method.

### EXAMPLES

The present invention will hereinafter be described more specifically by Examples. It should however be borne in mind that the present invention is not limited to or by the below-described Examples.

<Measuring Method of Solubility of Methyleneadamantane in Organic Compound>

To a predetermined amount of an organic solvent was added 2-methyleneadamantane in such an amount that it remains in the solvent after dissolution. After stirring the mixture while keeping the temperature at 0° C., the resulting mixture was allowed to stand. A supernatant was collected therefrom and its composition was analyzed by gas chromatography.

<Gas Chromatography Analysis>

The solubility of 2-methyleneadamantane and the amount of 2-methyleneadamantane in the reaction product were measured by gas chromatography analysis described below.
Gas chromatography analyzer: “GC-14B”, product of Shimadzu Corporation
Column: “TC-1”, product of Tokyo Chemical Industry (inner diameter: 0.25 mm, thickness of filler: 0.25 μm, length: 60 m)
Feed port temperature: 150° C.
Detector: FID
Detector temperature: 280° C.
Conditions of column oven: The column oven was retained at 100° C. for 3 minutes, heated to 180° C. at 10° C./min, retained at 180° C. for 5 minutes, heated to 280° C. at 10° C./min, and then retained at 280° C. for 5 minutes.

### Example 1

A reactor made of glass and equipped with a stirrer was charged with 9 L of toluene, 950 g of 2-methyladamantanol, 50 g of methacrylic acid and 5.3 g of cresol sulfonic acid. The resulting mixture was heated until the reflux of toluene occurred, followed by reflux for 2 hours while distilling off water, whereby 2-methyleneadamantane was prepared. After it was cooled to 50° C. and 8 L of toluene was distilled off from it under reduced pressure, the residue was cooled to 0° C. A methacrylate forming reaction was then caused by adding 1280 g of methacrylic acid and 83 g of boron trifluoride ethyl ether and stirring the resulting mixture for one hour. After the reaction mixture was washed with 6 L of a 10% aqueous sodium carbonate solution and 5 L of deionized water, toluene was distilled off at 70° C. under reduced pressure, whereby 1126 g of crude 2-methyladamantan-2-yl methacrylate was obtained. The resulting product contained 91.3 wt. % of 2-methyladamantan-2-yl methacrylate and 7.9 wt. % of 2-methyleneadamantane.

The crude 2-methyladamantan-2-yl(meth)acrylate (100 g) thus obtained and 50 g of n-undecane (boiling point under atmospheric pressure: 194.5° C., coagulation point: −25.6° C., solubility of methyleneadamantane in 100 g of n-undecane at 0° C.: 101 g) were distilled at 60° C. and 1 mmHg by using a vacuum still and 61 g was distilled off in 60 minutes. The fraction thus distilled off contained 86% of 2-methyleneadamantane together with undecane. The distillation temperature was raised to 85° C. and 3.5 g was distilled off further in 10 minutes to adjust the amount of 2-methyleneadamantane remaining in the still to less than 1 wt. %. The residue in the still was then distilled at 125° C. and 1 mmHg, whereby 81.2 g of 2-methyladamantan-2-yl(meth)acrylate having a purity of 99.5 wt. % was obtained. The resulting product had a 2-methyleneadamantane content of 0.1 wt. %.

### Example 2

In a similar distillation operation to that employed in Example 1 except that 50 g of n-tridecane (boiling point under atmospheric pressure: 234° C.) was charged instead of n-undecane and distillation was conducted at 60° C. and 1 mmHg, whereby 63.8 g was distilled off in 60 minutes. The resulting fraction thus distilled off contained 96% of 2-methyleneadamantane together with n-tridecane. The distillation temperature was raised to 90° C. and 3.2 g was distilled off further in 10 minutes. The residue in the still was then distilled at 125° C. and 1 mmHg to obtain a target substance, whereby 80.5 g of 2-methyladamantan-2-yl methacrylate having a purity of 99.6 wt. % was obtained. The resulting product had a 2-methyleneadamantane content of 0.1 wt. %.

### Example 3

In a similar distillation operation to that employed in Example 1 except that 75 g of n-decane (boiling point under atmospheric pressure: 174° C.) was charged instead of n-undecane, distillation was conducted at 60° C. and 1 mmHg, whereby 80.8 g was distilled off in 80 minutes. The resulting fraction thus distilled off contained 93% of 2-methyleneadamantane together with n-decane. The distillation temperature was raised to 90° C. and 7.3 g was distilled off in 15 minutes to decrease the amount of 2-methyleneadamantane to less than 1 wt. %. The residue in the still was then distilled at 125° C. and 1 mmHg to obtain a target substances whereby 79.6 g of 2-methyladamantan-2-yl methacrylate having a purity of 99.6 wt. % was obtained. The resulting product had a 2-methyleneadamantane content of 0.1 wt. %.

### Example 4

In a similar reaction operation to that employed in Example 1, 2-methyleneadamantane was prepared. After it was cooled to 50° C. and 8 L of toluene was distilled off under reduced pressure, the residue was cooled to 0° C. An acrylate forming reaction was then carried out by adding 1070 g of acrylic acid and 83 g of boron trifluoride ethyl ether to it while stirring the resulting mixture for one hour. After the reaction mixture was washed with 6 L of a 10% aqueous sodium carbonate solution and 5 L of deionized water, toluene was distilled off at 70° C. under reduced pressure, whereby 1050 g of crude 2-methyladamantan-2-yl acrylate was obtained. The resulting product contained 86.8 wt. % of 2-methyladamantan-2-yl acrylate and 12.5 wt. % of 2-methyleneadamantane. The crude 2-methyladamantan-2-yl acrylate (100 g) thus obtained and 50 g of n-undecane were charged in a vacuum still and distilled at 60° C. and 1 mmHg, whereby 63.3 g was distilled off in 60 minutes. The fraction thus distilled off contained 87% of 2-methyleneadamantane together with undecane. The distillation temperature was raised to 85° C. and 3.5 g was distilled out in 10 minutes to decrease the amount of 2-methyleneadamantane to less than 1 wt. %. The residue in the still was then distilled at 125° C. and 1 mmHg to obtain the target compound, whereby 785 g of 2-methyladamantan-2-yl acrylate having a purity of 99.5 wt. % was obtained. The resulting product had a 2-methyleneadamantane content of 0.1 wt. %.

### Comparative Example 1

In a similar distillation operation to that employed in Example 1 except that n-undecane was replaced by 200 g of deionized water, distillation was carried out at a distillation temperature of from 60° C. to 80° C. under reduced pressure at from 150 mmHg to 350 mmHg. As a result, it took 20 hours to reduce the amount of 2-methyleneadamantane in the residue in the still to less than 1%.

### Example 5

### Reaction Using a Mixed Solvent of Toluene and Undecane

A reactor made of glass was charged with 110 g of toluene, 50 g of n-undecane, 80 g of 2-methyladamantanol, 4.1 g of methacrylic acid and 0.92 g of p-toluenesulfonic monohydrate. The resulting mixture was heated until the reflux of toluene occurred, followed by reflux for 2 hours while distilling off water, whereby 2-methyleneadamantane was prepared it was then cooled to 0° C. A methacrylate forming reaction was carried out by adding 103.6 g of methacrylic acid and 13.7 g of boron trifluoride ethyl ether to it and stirring the resulting mixture for 2 hours. The reaction mixture was washed with 1275.5 g of a 10% aqueous sodium carbonate solution, followed by washing three times with 160 g of deionized water. Toluene was then distilled off at 70° C. under reduced pressure, whereby 148.8 g of crude 2-methyladamantan-2-yl methacrylate was obtained. The resulting product contained 59.9 wt. % of 2-methyladamantan-2-yl methacrylate and 7.6 wt. % of 2-methyleneadamantane.

The crude 2-methyladamantan-2-yl methacrylate was distilled at 60° C. and 1 mmHg by using a vacuum still and 59 g was distilled off in 60 minutes. The fraction thus distilled off contained 87% of 2-methyleneadmanatane together with n-undecane. The distillation temperature was raised to 85° C. and 3.4 g was distilled out in 10 minutes to adjust the amount of 2-methyleneadamantane remaining in the residue in the still to less than 1 wt. %. The residue in the still was then distilled at 125° C. and 1 mmHg, whereby 79.1 g of 2-methyladamantan-2-yl methacrylate having a purity of 99.4 wt. % was obtained. The resulting product had a 2-methyleneadamantane content of 0.1 wt. %.

### Example 6

### Reaction Using an Undecane Solvent

A reactor made of glass was charged with 160 g of n-undecane, 80 g of 2-methyladamantanol, 4.1 g of methacrylic acid and 0.92 g of p-toluenesulfonic monohydrate. The resulting mixture was maintained for 1 hour while distilling off water under atmospheric pressure. Water was removed further by reflux at 120° C. for 1 hour under reduced pressure at 150 mmHg, whereby 2-methyladamantane was prepared. It was cooled to 0° C. A methacrylate forming reaction was then carried out by adding 103.6 g of methacrylic acid and 13.7 g of boron trifluoride ethyl ether to it while stirring the resulting mixture for 2 hours. After the reaction mixture was washed with 1275.5 g of a 10% aqueous sodium carbonate solution, followed by washing three times with 160 g of deionized water. In such a manner, 260 g of a n-undecane solution of 2-methyladamantan-2-yl methacrylate was obtained. The resulting solution contained 29.6 wt. % of 2-methyladamantan-2-yl methacrylate and 5.2 wt. % of 2-methyleneadamantane.

The present invention was described specifically by using specific modes. It is however apparent for those skilled in the art that the present invention can be changed or modified without departing from the gist and scope of the present invention.

### INDUSTRIAL APPLICABILITY

The present invention makes it possible to prepare target 2-methyladamantan-2-yl(meth)acrylate with high separation efficiency from a mixture obtained by reacting 2-methyleneadamantane with (meth)acrylic acid in the presence of an acid catalyst.

1. A process for preparing 2-methyladamantan-2-yl(meth)acrylate, which comprises: reacting 2-methyleneadamantane and (meth)acrylic acid by an acid catalyst to obtain a reaction mixture; and separating 2-methyleneadamantane from the reaction mixture to obtain 2-methyladamantan-2-yl(meth)acrylate, wherein the reaction mixture is distilled in the presence of an organic compound having a boiling point, under atmospheric pressure, of 150° C. or greater but not greater than 260° C. and immiscible with water.

2. The process for preparing 2-methyladamantan-2-yl(meth)acrylate according to claim 1, wherein the organic compound has a coagulation point of 10° C. or less.

3. The process for preparing 2-methyladamantan-2-yl(meth)acrylate according to claim 1, wherein the solubility at 0° C. of 2-methyladamantane in 100 g of the organic compound is 5 g or greater.

4. The process for preparing 2-methyladamantan-2-yl(meth)acrylate according to claim 1, wherein the organic compound is a hydrocarbon compound.

5. The process for preparing 2-methyladamantan-2-yl(meth)acrylate according to claim 4, wherein the hydrocarbon compound is an aliphatic hydrocarbon.

6. A process for preparing 2-methyladamantan-2-yl(meth)acrylate, which comprises:
(a) reacting 2-methyleneadamantane with (meth)acrylic acid by an acid catalyst to obtain a mixture containing 2-methyleneadamantane and 2-methyladamantan-2-yl(meth)acrylate;
(b) distilling the reaction mixture in the presence of an organic compound having a boiling point, under atmospheric pressure of 150° C. or greater but not greater than 260° C. and immiscible with water to distill off a fraction comprising 2-methyleneadamantane as a main component from the top of a distillation column and to obtain a fraction comprising 2-methyladamantan-2-yl(meth)acrylate as a main component from the bottom of the distillation column; and
(c) re-distilling the fraction obtained from the bottom of the distillation column and comprising 2-methyladamantan-2-yl(meth)acrylate as a main component to remove the organic compound having a boiling point of 150° C. or greater but not greater than 260° C., 2-methyleneadamantane and a portion of 2-methyladamantan-2-yl(meth)acrylate from the top of the distillation column to reduce the amount of 2-methyleneadamantane present in 2-methyladamantan-2-yl(meth)acrylate to 1 wt. % or less.

7. The process for preparing 2-methyladamantan-2-yl(meth)acrylate according to claim 6, wherein at least a portion of the fraction distilled off from the top of the distillation column in (c) is recycled to (b).

8. A process for preparing 2-methyladamantan-2-yl(meth)acrylate which comprises: reacting 2-methyleneadamantane and (meth)acrylic acid in the presence of an organic compound having a boiling point, under atmospheric pressure of 150° C. or greater but not greater than 260° C. and immiscible with water by an acid catalyst to obtain a reaction mixture; and separating 2-methyladamantane from the reaction mixture by distillation to obtain 2-methyladamantan-2-yl(meth)acrylate.

9. A process for preparing 2-methyladamantan-2-yl(meth)acrylate, which comprises:
(a) reacting 2-methyleneadamantane with (meth)acrylic acid by an acid catalyst to obtain a mixture containing 2-methyleneadamantane and 2-methyladamantan-2-yl(meth)acrylate;
(b) distilling the reaction mixture in the presence of an organic compound having a boiling point, under atmospheric pressure, of 150° C. or greater but not greater than 260° C. and immiscible with water, to distill off a fraction comprising 2-methyleneadamantane as a main component from the top of a distillation column and to obtain a fraction comprising 2-methyladamantan-2-yl(meth)acrylate as a main component from the bottom of the distillation column; and
(c) recycling, to (a), at least a portion of the fraction containing 2-methyleneadamantane and the organic compound having a boiling point, under atmospheric pressure, of 150° C. or greater but not greater than 260° C. and immiscible with water.

10. 2-Methyladamantan-2-yl(meth)acrylate having a 2-methyleneadamantane content of 1 wt. % or less.
